(19) United States
(12) Patent Application Publication
(10) Pub. No.: US 2026/0112462 A1
(43) Pub. Date: Apr. 23, 2026

(54) SYSTEMS AND METHODS FOR IDENTIFYING PREDICTIVE PARAMETERS BASED ON RESULTS OF CLINICAL STUDIES

(71) Applicant: Form Bio Inc., Austin, TX (US)

(72) Inventors: Andrew Busey, Austin, TX (US); Amicia Devin Elliott, Burien, WA (US); Claire Aldridge, Dallas, TX (US)

(21) Appl. No.: 18/164,877

(22) Filed: Feb. 6, 2023

(51) Int. Cl. G16H 10/20; G06N 20/00

(52) U.S. Cl. CPC G16H 10/20; G06N 20/00

(57) ABSTRACT

A system and method for identifying predictive parameters based on results of clinical studies are disclosed. Exemplary implementations may: obtain clinical information that includes responses by individual subjects of a first set of subjects to being administered a first intervention; identify subsets of individual subjects within the first set of subjects based on the responses; obtain subject information for the individual subjects of the first set of subjects, the subject information including parameter values for one or more biological parameters and historical parameters; identify commonality of parameter values for individual parameters included in the subject information within the individual identified subsets; provide the parameters for which commonality of parameter values within the subsets are identified; and/or other exemplary implementations.

### FIELD OF THE DISCLOSURE

The present disclosure relates to systems and methods for identifying predictive parameters based on results of clinical studies.

### BACKGROUND

Methods for analyzing distribution data are known. Methods for analyzing biological and genomic information are known (e.g., DNA sequencing). Analysis of biological and genomic information may include determining biological profiles (e.g., phenotypes, genotypes, etc.).

### SUMMARY

Clinical studies may be conducted with human subjects to evaluate the effects and/or efficacy of different types of interventions. The interventions may include different types of drug or gene therapies, behavioral interventions, medical procedures, and/or other types of interventions. Results of clinical studies may be assessed based on the outcome disparity between a treatment group that receives the intervention and a control group that does not receive the intervention. In some cases, outcomes between the treatment and control groups may lack statistical significance. In other words, the effects and/or responses of the human subjects are unable to be attributed to the intervention being evaluated. The responses of the human subjects may be analyzed to identify parameters that are correlated to the responses. Parameters may include aspects of the human subjects'genomic information and/or other information. Identification of correlated parameters may be used to generate predictive models for other subjects and/or studies. In some cases, the identification of correlated parameters may aid in modification of the clinical studies (e.g., to account for the correlated parameter). Overall, the identification of correlated parameters may provide researchers with another level of analysis to enhance the efficacy of clinical studies.

One or more aspects of the present disclosure include a system for identifying predictive parameters based on results of clinical studies. The system may include one or more hardware processors configured by machine-readable instructions, electronic storage, and/or other components. Executing the machine-readable instructions may cause the one or more hardware processors to update a trained machine learning model to generate alternative nucleic acid sequences. The machine-readable instructions may include one or more computer program components. The one or more computer program components may include one or more of an information component, an identification component, a subject component, a parameter component, an output component, a model component, and/or other components.

The information component may be configured to obtain clinical information and/or other information. The clinical information may correspond to a first study conducted on a first set of subjects. The first study may be conducted to evaluate a first intervention. The clinical information may include responses by individual subjects of the first set of subjects to being administered the first intervention and/or other information.

The identification component may be configured to identify subsets of individual subjects within the first set of subjects based on the responses. Individual subjects of a given subset may share the same or similar response to being administered the first intervention. By way of non-limiting illustration, a first subset and a second subset may be identified. The first subset may include individual subjects having responses that are the same or similar to each other to being administered the first intervention. The second subset may include individual subjects having responses that are the same or similar to each other to being administered the first intervention. The responses of the subjects of the first subset may be different and distinct from the responses of the subjects of the second subset.

The subject component may be configured to obtain subject information for the individual subjects of the first set of subjects and/or other information. The subject information may characterize aspects of the individual subjects relevant to the responses to being administered the first intervention. By way of non-limiting illustration, the subject information for a given subject of the first set of subjects may include parameter values for one or more biological parameters related to the biological profile of the given subject, one or more historical parameters related to the medical history of the given subject, and/or parameter values for one or more other types of parameters.

The parameter component may be configured to identify commonality of parameter values for individual parameters included in the subject information within the individual identified subsets. The identification may be based on the subject information, the identified subsets of subjects, and/or other information. By way of non-limiting illustration, commonality of parameter values for a first parameter may be identified for the first subset and commonality of parameter values for a second parameter may be identified for the second subset.

The output component may be configured to provide the parameters for which commonality of parameter values within the subsets are identified. By way of non-limiting illustration, the first parameter may be provided with respect to the first subset, and the second parameter may be provided with respect to the second subset.

These and other features, and characteristics of the present technology, as well as the methods of operation and functions of the related elements of structure and the combination of parts will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRAWINGS

FIG. 1 illustrates a system for identifying predictive parameters based on results of clinical studies, in accordance with one or more implementations.

FIG. 2 illustrates a method for identifying predictive parameters based on results of clinical studies, in accordance with one or more implementations.

FIGS. 3A-B illustrate an exemplary user interface, in accordance with one or more implementations.

### DETAILED DESCRIPTION

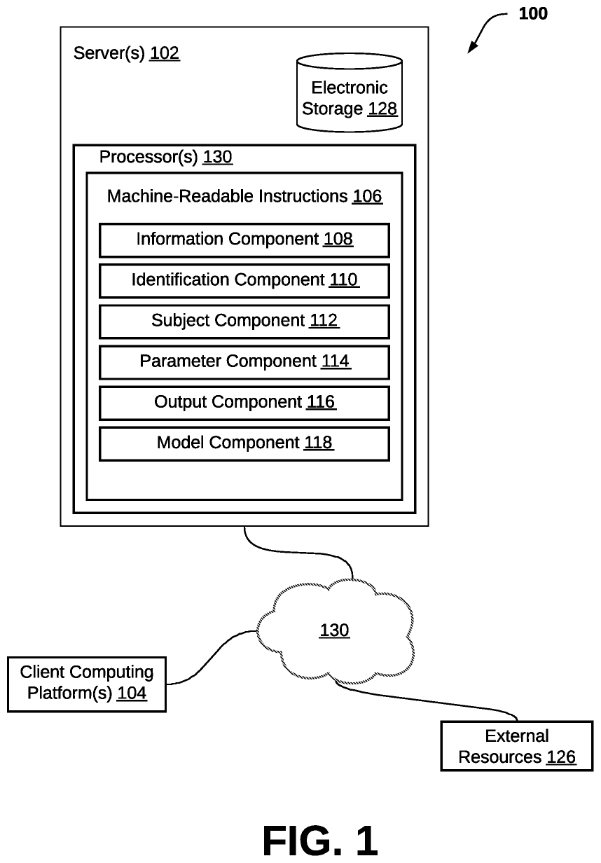

FIG. 1 illustrates a system 100 configured to identify predictive parameters based on results of clinical studies, in accordance with one or more implementations. In some implementations, system 100 may include one or more servers 102. Server(s) 102 may include electronic storage 128, one or more processors 130, and/or other components. Server(s) 102 may be configured to communicate with one or more client computing platforms 104 according to a client/server architecture and/or other architectures. Client computing platform(s) 104 may be configured to communicate with other client computing platforms via server(s) 102 and/or according to a peer-to-peer architecture and/or other architectures. Users may access system 100 via client computing platform(s) 104.

Server(s) 102 may be configured by machine-readable instructions 106. Machine-readable instructions 106 may include one or more instruction components. The instruction components may include computer program components. The instruction components may include one or more of information component 108, identification component 110, subject component 112, parameter component 114, output component 116, model component 118, and/or other instruction components.

Information component 108 may be configured to obtain clinical information and/or other information. The clinical information may correspond to a first study conducted on a first set of subjects. The first study may be conducted to evaluate a first intervention. In some implementations, the first study may have been conducted to evaluate the effect of the first intervention on human test subjects. The clinical information may include responses by individual subjects of the first set of subjects to being administered the first intervention and/or other information. In some implementations, the first intervention may be a drug therapy, a medical device, a medical procedure, a vaccine, and/or other types of interventions.

Individual subjects of the first set of subjects may be sorted (i.e., assigned) into one or more clinical groups for the first study. The clinical groups may include one or more of treatment groups, control groups, and/or other types of groups of subjects. In some implementations, the clinical groups may determine whether a given subject will be administered the first interventions and/or what intervention the given subject may be administered. By way of non-limiting illustration, individual subjects of the first set of subjects may be assigned to a first treatment group or a first control group. Assignment of individuals of the first set of subjects to the first treatment group or the first control group may be randomly determined. Individual subjects of the first treatment group may be separate and distinct from individual subjects of the first control group. The clinical information may include responses from individuals of the first treatment group, responses from individuals of the first control group, and/or other information. The responses from individuals of the first treatment group may be in response to being administered the first intervention. The responses from individuals of the first control group may be in response to being administered a placebo for the first intervention and/or not being administered an intervention. In some implementations, responses from individuals of the first treatment group may be similar to responses from individuals of the first control group. Responsive to the similarity between responses of individuals of the first treatment group and the responses of the individuals of the first control group meeting or exceeding a threshold, the results of the first study may be determined to be statistically insignificant. In other words, the results of the first study may not be attributable to the effects of the first intervention.

In some implementations, the clinical information may include information related to the methodology and/or execution of the first study. By way of non-limiting illustration, information related to the methodology and execution of the first study may describe protocols (e.g., U.S. Food and Drug Administration regulations, methods for measuring responses, determination and/or treatment of treatment groups, control groups, etc.), costs (i.e., budgets, funding), subject requirements, the first intervention (e.g., side effects, risks, etc.), and/or other information pertaining to conducting the first study. In some implementations, the clinical information may be obtained from electronic storage 128, external resources 126, and/or other components of system 100. The clinical information may be obtained, responsive to information component 108 providing validation of permission to access, view, and/or edit the clinical information. Validation may include a digital token, public and/or private key, authentication key, and/or other methods of digital certification. By way of non-limiting illustration, failure to provide validation of permission to access the clinical information may prevent information component 108 (and/or other components of system 100) from obtaining the clinical information.

Identification component 110 may be configured to identify subsets of individual subjects within the first set of subjects based on the responses. By way of non-limiting illustration, a first subset and a second subset may be identified. The first subset may include individual subjects having responses that are the same or similar to each other to being administered the first intervention. The second subset may include individual subjects having responses that are the same or similar to each other to being administered the first intervention. The responses of the subjects of the first subset may be different and distinct from the responses of the subjects of the second subset.

In some implementations, subsets of individual subjects within the first set of subjects may be identified responsive to a determination that the results of the first study are statistically insignificant (i.e., lacking statistical significance). Individual subjects of a given subset may share the same or similar responses. The responses may be to one or more of being administered the first intervention, being administered a placebo for the first intervention, and/or not being administered an intervention. The given subset may include individuals assigned to one or more different clinical groups (e.g., the first treatment group and the first control group) at the start of the first study. By way of non-limiting illustration, the given subset may include individuals assigned to the first treatment group and may include individuals assigned to the first control group. The responses of individual subjects of the first set of subjects may include values (and/or ranges of values) of one or more of biological measures, observed behaviors, and/or other types of responses exhibited by subjects. Biological measures may include hormone levels, presence and/or levels of proteins and/or compounds (e.g., white blood cell count, antibodies, protein markers, etc.), and/or other types of biological measures. In some implementations, the given subset may be characterized by a range of values for one or more types of responses. By way of non-limiting illustration, the given subset may be characterized by a first range of values for a first type of response. Individual subjects of the first set of subjects having responses within the first range of values for the first type of response may be included in the given subset. Individual subjects of the first set of subjects having responses outside the first range of values for the first type of response may be included in other subsets and/or may not be included in the given subset.

In some implementations, the subsets may be identified to accordance with one or more subset requirements. Subset requirements may be selected by one or more users via client computing platforms 104 and/or determined by identification component 110. The subset requirements may include one or more limitations and/or constraints on the identification of the subsets. By way of non-limiting illustration, a first subset requirement may limit the number of subsets identified to two subsets, three subsets, and/or other numbers of subsets. A second subset requirements may describe a limit for the ranges of values of responses that characterizes the subset. A third subset requirement may specify a type of response for identifying subsets of subjects. The mentioned subset requirements are exemplary descriptions of subset requirements and are not intended to be limiting.

Subject component 112 may be configured to obtain subject information for the individual subjects of the first set of subjects and/or subjects. The subject information may characterize aspects of the individual subjects of the first set of subjects relevant to the responses. The responses may be to the administration of the first intervention, being administered a placebo for the first intervention, and/or not being administered an intervention. The subject information for a given subject of the first of subjects may include one or more parameter values for one or more biological parameters related to the biological profile of the given subject, one or more historical parameters related to the medical history of the given subject. Biological parameters may include the age, sex, ancestry, phenotypes, genotypes, and/or other biological information pertaining to the given subject. In some implementations, biological parameters may include biometric parameters. By way of non-limiting illustration, biometric parameters may include heart rate, glucose level, sleep patterns, and/or other types of biological measurements associated with the subjects. The values of the biometric parameters may be obtained via subject input and/or recorded via wearable devices associated with (e.g., worn by) the subjects. By way of non-limiting illustration, wearable devices capable of measuring values of biometric parameters may include smart watches, pedometers, heart rate monitors, and/or other types of wearable devices. Historical parameters may include illnesses, allergies, immunizations, family medical history, surgical interventions, prior medications, and/or other types of historical parameters pertaining to the given subject. In some implementations, historical parameters may include environmental exposures associated with the subjects. By way of non-limiting illustration, environmental exposures may include exposure to hazardous waste, water contamination, and/or other information pertaining to the locations of the subjects.

In some implementations, access permissions are required to the subject information for the first set of subjects and/or other sets of subjects. In some implementations, the subject information may be obtained from electronic storage 128, external resources 126, and/or other components of system 100. The subject information may be obtained, responsive to information component 108 providing validation of permission to access, view, and/or edit the subject information. Validation may include a digital token, public and/or private key, authentication key, and/or other methods of digital certification. In some implementations, validation may include proof of consent (e.g., release forms, waivers, disclosures, etc.) by a given subject to access, view, and/or edit the subject information for the given subject. By way of non-limiting illustration, failure to provide validation of permission to access the subject information may prevent subject component 112 (and/or other components of system 100) obtaining the subject information.

Parameter component 114 may be configured to identify commonality of parameter values for individual parameters included in the subject information within the individual identified subsets. The commonality of parameters values may be for one or more biological parameters, historical parameters, and/or other types of parameters. The identification may be based on the subject information, the identified subsets of subjects, and/or other information. By way of non-limiting illustration, commonality of parameter values for a first parameter may be identified for the first subset and commonality of parameter values for a second parameter may be identified for the second subset. In some implementations, commonality of parameter values may be defined by a range of values. By way of non-limiting illustration, the subject information for individual subjects of the first subset may include values for the first parameter that are within a first range. The subject information for individual subjects of the second subset may include values for the second parameter that are within a second range. The commonality of parameter values for the first parameter may indicate the subject information for individual subjects of the first subset include a first value and/or a first range of values for the first parameter.

The commonality of parameter values for the first parameter may be identified responsive to a number of subjects of the first subset sharing and/or having similar parameter values for the first parameter meeting or exceeding a threshold. The commonality of parameter values for the second parameter may be identified responsive to a number of subjects of the second subset sharing and/or having similar parameter values for the second parameter. A commonality of parameter values for a given parameter may be identified for an identified given subset. The commonality of parameter values may be identified responsive to a number of subjects of the given subset sharing and/or having similar parameter values for the given parameter. In some implementations, the commonality of parameter values may be identified responsive to the number of subjects that share and/or have similar parameter values for the given parameter meeting or exceeding a threshold. The threshold may be defined by a number of subjects, a portion and/or percentage of the subjects, and/or other types of values. The portion and/or percentage of the subjects that defines the threshold may be relative to the number of subjects in the first set of subjects and/or the number of subjects in the given subset. By way of non-limiting illustration, the commonality of parameter values for the given parameter may be identified responsive to 80% of the subjects within the given subset sharing and/or having similar parameter values for the given parameter.

In some implementations, the commonality of parameter values may be identified responsive to the number of subjects within the given subset sharing the parameter values and a number of subjects outside the given subset lacking the same and/or similar parameter values. By way of non-limiting illustration, the commonality of parameter values for the given parameter for the given subset may be identified response to 90% of the subjects outside the given subset having a parameter value for the given parameter that is not the same as and/or outside the range of the commonality of the parameter value for the given parameter. In some implementations, the commonality of parameter values for the individual subsets may be identified responsive to a determination that the parameter is statistically significant to the results of the first study (i.e., the responses of individual subjects in the first set of subjects). In other words, the responses of individual subjects in the first set of subjects may be associated with the values of the parameter for which commonality of parameter values are identified.

The commonality of parameter values for the identified subsets within the first set of subjects may be used to determine response predictions for individual subjects of a second set of subjects. The individual subjects of the second set of subjects may have not been administered the first intervention and/or otherwise participated in the first study and/or other studies. The second set of subjects may include a first subject, a second subject, and/or other subjects. In some implementations, subject information for the first subject including the commonality of parameter values for the first parameter may be predictive of the first subject having a response to being administered the first intervention that is the same or similar to the individual subjects of the first subset. Subject information for the second subject including the commonality of parameter values for the second parameter may be predictive of the second subject having a response to being administered the first intervention that is the same or similar to the individual subjects of the second subset.

Output component 116 may be configured to provide the parameters for which commonality of parameter values within the subsets are identified. By way of non-limiting illustration, the first parameter may be provided with respect to the first subset, the second parameter may be provided with respect to the second subset, and so on and so forth. The first parameter may be provided with the identified commonality of parameter value and/or range of values within the first subset. The second parameter may be provided with the identified commonality of parameter value and/or range of values within the second subset. In some implementations, output component 116 may be configured to generate a clinical study report and/or other documents. The clinical study report may be based on the provided parameters for which commonality of parameter values within the subsets are identified. The clinical study report may describe the commonality of parameters values, the identified subsets, the responses of individual subjects within the same subsets, and/or other information. The clinical study report may describe the statistical relationship between the identified commonality of parameter values within the identified subsets and the responses of individual subjects of the identified subsets. By way of non-limiting illustration, the clinical study report may describe the relationship between the identified commonality of the parameter value (of the first parameter) within the first subset and the responses of individual subject of the first subset.

In some implementations, subject component 112 may be configured to obtain subject information for a second set of subjects. The subjects of the second set of subjects may have not been administered the first intervention. Parameter component 114 may be configured to identify individual subjects of the second set of subjects corresponding to subject information that includes the commonality of parameter values for parameters identified for the first set of subjects. In some implementations, parameter component 114 may be configured to determine response predictions for the individual subjects of the second set of subjects. The response predictions may be based on the commonality of parameter values for parameters identified for the first set of subjects, the subject information for subjects of the second set of subjects, and/or other information. The response predictions for the individual subjects of the second set of subjects may describe the expected response of the subjects to being administered the first intervention.

Model component 118 may be configured to aggregate the commonality of parameter values and the responses of subjects of the corresponding subsets into training input/output pairs. Individual training input/output pairs may include training input information, training output information, and/or other information. By way of non-limiting illustration, model component 118 may be configured to aggregate a first input/output pair, a second input/output pair, and/or other input/output pairs. The first input/output pair may include first training input information and first training output information. The first training input information may be the common parameter values for the first parameter. The first training output information may be the shared response of individual subjects of the first subset. The second input/output pair may include second training input information and second training output information. The second training input information may be the common parameter values for the second parameter. The second training output information may be the shared response of individual subjects of the second subset.

Model component 118 may be configured to train a machine learning model based on the aggregated training input/output pairs. The trained model may thereafter be configured to automatically generate response predictions for subjects based on subject information for the subjects, identified commonality of parameters values for subsets of other subjects, and/or other information. The response predictions may be the expected response from a given subject to being administered the first intervention. In some implementations, the machine learning model may utilize one or more of an artificial neural network, naïve bayes classifier algorithm, k means clustering algorithm, support vector machine algorithm, linear regression, logistic regression, decision trees, random forest, nearest neighbors, and/or other approaches. Model component 118 may utilize training techniques such as supervised learning, semi-supervised learning, unsupervised learning, reinforcement learning, and/or other techniques.

In supervised learning, the model may be provided with known training dataset that includes desired inputs and outputs (e.g., the input/output pairs described herein), and the model may be configured to find a method to determine how to arrive at those outputs based on the inputs. The model may identify patterns in data, learn from observations, and make predictions. The model may make predictions and may be corrected by an operator—this process may continue until the model achieves a high level of accuracy/performance. Supervised learning may utilize approaches including one or more of classification, regression, and/or forecasting.

Semi-supervised learning may be similar to supervised learning, but instead uses both labelled and unlabeled data. Labelled data may comprise information that has meaningful tags so that the model can understand the data (e.g., the input/output pairs described herein), while unlabeled data may lack that information. By using this combination, the machine learning model may learn to label unlabeled data.

For unsupervised learning, the machine learning model may study data to identify patterns. There may be no answer key or human operator to provide instruction. Instead, the model may determine the correlations and relationships by analyzing available data. In an unsupervised learning process, the machine learning model may be left to interpret large data sets and address that data accordingly. The model tries to organize that data in some way to describe its structure. This might mean grouping the data into clusters or arranging it in a way that looks more organized.

Unsupervised learning may use techniques such as clustering and/or dimension reduction.

Reinforcement learning may focus on regimented learning processes, where the machine learning model may be provided with a set of actions, parameters, and/or end values. By defining the rules, the machine learning model then tries to explore different options and possibilities, monitoring and evaluating each result to determine which one is optimal to generate correspondences. Reinforcement learning teaches the model trial and error. The model may learn from past experiences and adapt its approach in response to the situation to achieve the best possible result.

Model component 118 may be configured to store the trained machine learning model. In some implementations, the trained machine learning model (i.e., “the model”) may be stored in electronic storage 128.

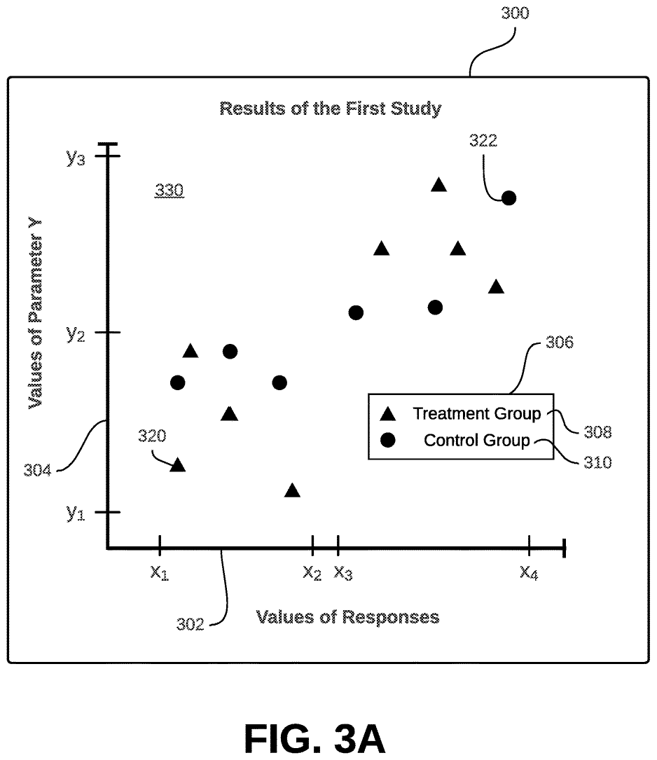

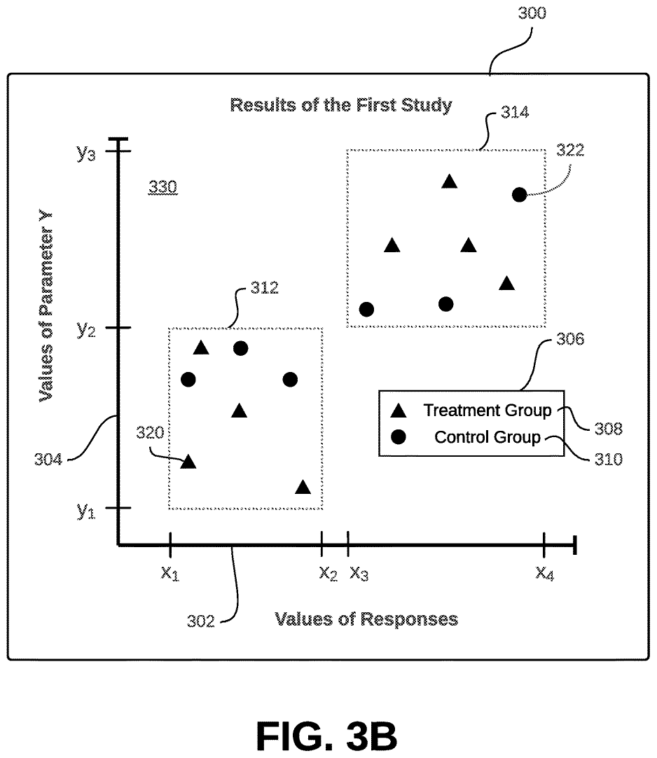

FIGS. 3A-B illustrate an exemplary user interface 300. User interface 300 may include a graphical representation 330 of results of a first study. The first study may be conducted on a first set of subjects to evaluate a first intervention. Graphical representation 300 is shown as a plot graph, however this is not intended to be limiting. Graphical representation 300 may include one or more of a first axis 302, a second axis 304, graph key 306, and/or other graphical elements. First axis 302 may correspond to values of responses by subjects in the first set of subjects and/or other information. The responses by individual subjects in the first set of subjects may be in response to being administered the first intervention, being administered a placebo for the first intervention, and/or not being administered an intervention. Second axis 304 may correspond to values of a parameter Y and/or other information. Parameter Y may be a biological parameter related to the biological profile of the given subject, a historical parameter related to the medical history of the given subject, and/or other types of parameters. Graph key 306 may include a first icon for a treatment group 308 (represented by a triangular icon), a second icon for a control group 310 (represented by a circular icon), and/or other types of icons. Individual subjects of the first set of subjects may be randomly assigned to the treatment group or the control group within the first study. In some implementations, individual subjects assigned to the treatment group may be administered the first intervention. Individual subjects assigned to the control group may be administered a placebo for the first intervention and/or not be administered the first intervention.

Referring to FIG. 3A, graphical representation 330 include one or more plots and/or other graphical elements. Individual plots included in graphical representation 330 may correspond to individual subjects of the first set of subjects. By way of limiting illustration, graphical representation 330 may include a first plot point 320, a second plot point 322, and/or other graphical elements. First plot point 320 may correspond to a first subject in the first set of subjects. First plot point 320 being a triangular icon may indicate the first subject being assigned to the treatment group and/or being administered the first intervention within the first study. Second plot point 322 may correspond to a second subject in the first set of subjects. Second plot point 322 being a circular icon may indicate the second subject being assigned to the control group and/or being administered a placebo for the first intervention within the first study.

Referring to FIG. 3B, graphical representation 330 may include one or more graphical elements to indicate one or more identified subsets of subjects. The graphical elements may include a first indicator 312 for a first identified subset, a second indicator 314 for a second identified subset, and/or other graphical elements. First indicator 312 and second indicator 314 are shown as dashed squares, it will be appreciated that this is not intended to be limiting. Individual subjects corresponding to plot points within first indicator 312 may be included in the first subset, and individual subjects corresponding to plot points within second indicator 314 may be included in the second subset. In some implementations, the first subset may be identified based on individual subjects sharing the same and/or similar responses within the first study. Responses of individual subjects of the first subset may be within a first range of response values (e.g., between x₁ and x₂). The second subset may be identified based on individual subjects sharing the same and/or similar responses within the first study. Responses of individual subjects of the second subset may be within a second range of response values (e.g., between x₃ and x₄). First indicator 312 may indicate identified commonality of parameter values for parameter Y within the first subset. The commonality of parameter values for parameter Y within the first subset may be defined by a first range of parameter values (e.g., between y₁ and y₂). Second indicator 314 may indicate identified commonality of parameter values for parameter Y within the second subset. The commonality of parameter values for parameter Y within the second subset may be defined by a second range of parameter values (e.g., between y₃ and y₄).

In some implementations, server(s) 102, client computing platform(s) 104, and/or external resources 126 may be operatively linked via one or more electronic communication links. For example, such electronic communication links may be established, at least in part, via a network 130 such as the Internet and/or other networks. It will be appreciated that this is not intended to be limiting, and that the scope of this disclosure includes implementations in which server(s) 102, client computing platform(s) 104, and/or external resources 126 may be operatively linked via some other communication media.

A given client computing platform 104 may include one or more processors configured to execute computer program components. The computer program components may be configured to enable an expert or user associated with the given client computing platform 104 to interface with system 100 and/or external resources 126, and/or provide other functionality attributed herein to client computing platform(s) 104. By way of non-limiting example, the given client computing platform 104 may include one or more of a desktop computer, a laptop computer, a handheld computer, a tablet computing platform, a Smartphone, and/or other computing platforms.

External resources 126 may include sources of information outside of system 100, external entities participating with system 100, and/or other resources. In some implementations, some or all of the functionality attributed herein to external resources 126 may be provided by resources included in system 100.

Server(s) 102 may include communication lines, or ports to enable the exchange of information with a network and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 128 may comprise non-transitory storage media that electronically stores information. The electronic storage media of electronic storage 128 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 128 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 128 may include one or more virtual storage resources (e.g., cloud storage, a virtual private network, and/or other virtual storage resources). Electronic storage 128 may store software algorithms, information determined by processor(s) 130, information received from server(s) 102, information received from client computing platform(s) 104, and/or other information that enables server(s) 102 to function as described herein.

Processor(s) 130 may be configured to provide information processing capabilities in server(s) 102. As such, processor(s) 130 may include one or more of a digital processor, an analog processor, a digital circuit designed to process information, an analog circuit designed to process information, a state machine, and/or other mechanisms for electronically processing information. Although processor(s) 130 is shown in FIG. 1 as a single entity, this is for illustrative purposes only. In some implementations, processor(s) 130 may include a plurality of processing units. These processing units may be physically located within the same device, or processor(s) 130 may represent processing functionality of a plurality of devices operating in coordination. Processor(s) 130 may be configured to execute components 108, 110, 112, 114, 116, and/or 118, and/or other components. Processor(s) 130 may be configured to execute components 108, 110, 112, 114, 116, and/or 118, and/or other components by software; hardware; firmware; some combination of software, hardware, and/or firmware; and/or other mechanisms for configuring processing capabilities on processor(s) 130. As used herein, the term “component” may refer to any component or set of components that perform the functionality attributed to the component. This may include one or more physical processors during execution of processor readable instructions, the processor readable instructions, circuitry, hardware, storage media, or any other components.

It should be appreciated that although components 108, 110, 112, 114, 116, and/or 118 are illustrated in FIG. 1 as being implemented within a single processing unit, in implementations in which processor(s) 130 includes multiple processing units, one or more of components 108, 110, 112, 114, 116, and/or 118 may be implemented remotely from the other components. The description of the functionality provided by the different components 108, 110, 112, 114, 116, and/or 118 described below is for illustrative purposes, and is not intended to be limiting, as any of components 108, 110, 112, 114, 116, and/or 118 may provide more or less functionality than is described. For example, one or more of components 108, 110, 112, 114, 116, and/or 118 may be eliminated, and some or all of its functionality may be provided by other ones of components 108, 110, 112, 114, 116, and/or 118. As another example, processor(s) 130 may be configured to execute one or more additional components that may perform some or all of the functionality attributed below to one of components 108, 110, 112, 114, 116, and/or 118.

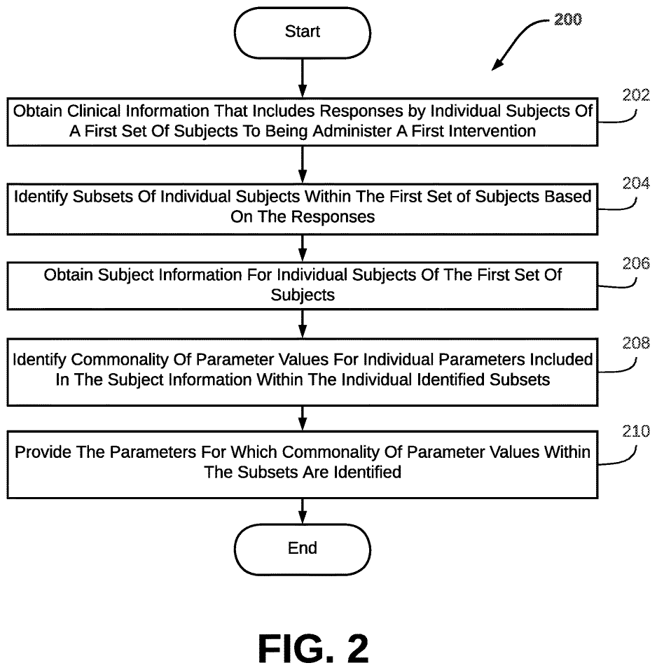

FIG. 2 illustrates a method 200 for identifying predictive parameters based on results of clinical studies, in accordance with one or more implementations. The operations of method 200 presented below are intended to be illustrative. In some implementations, method 200 may be accomplished with one or more additional operations not described, and/or without one or more of the operations discussed. Additionally, the order in which the operations of method 200 are illustrated in FIG. 2 and described below is not intended to be limiting.

In some implementations, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

An operation 202 may include obtaining clinical information and/or other information. The clinical information may correspond to a first study conducted on a first set of subjects. The first study may be conducted to evaluate a first intervention. The clinical information may include responses by individual subjects of the first set of subjects to being administered the first intervention and/or other information. Operation 202 may be performed by one or more hardware processors configured by machine-readable instructions including a component that is the same as or similar to information component 108, in accordance with one or more implementations.

An operation 204 may include identifying subsets of individual subjects within the first set of subjects based on the responses. Individual subjects of a given subset may share the same or similar response to being administered the first intervention. By way of non-limiting illustration, a first subset and a second subset may be identified. The first subset may include individual subjects having responses that are the same or similar to each other to being administered the first intervention. The second subset may include individual subjects having responses that are the same or similar to each other to being administered the first intervention. The responses of the subjects of the first subset may be different and distinct from the responses of the subjects of the second subset. Operation 204 may be performed by one or more hardware processors configured by machine-readable instructions including a component that is the same as or similar to identification component 110, in accordance with one or more implementations.

An operation 206 may include obtaining subject information for the individual subjects of the first set of subjects and/or other information. The subject information may characterize aspects of the individual subjects relevant to the responses to being administered the first intervention. By way of non-limiting illustration, the subject information for a given subject of the first set of subjects may include parameter values for one or more biological parameters related to the biological profile of the given subject, one or more historical parameters related to the medical history of the given subject, and/or parameter values for one or more other types of parameters. Operation 206 may be performed by one or more hardware processors configured by machine-readable instructions including a component that is the same as or similar to subject component 112, in accordance with one or more implementations.

An operation 208 may include identifying commonality of parameter values for individual parameters included in the subject information within the individual identified subsets. The identification may be based on the subject information, the identified subsets of subjects, and/or other information. By way of non-limiting illustration, commonality of parameter values for a first parameter may be identified for the first subset and commonality of parameter values for a second parameter may be identified for the second subset. Operation 208 may be performed by one or more hardware processors configured by machine-readable instructions including a component that is the same as or similar to parameter component 114, in accordance with one or more implementations.

An operation 210 may include providing the parameters for which commonality of parameter values within the subsets are identified. By way of non-limiting illustration, the first parameter may be provided with respect to the first subset, and the second parameter may be provided with respect to the second subset. Operation 210 may be performed by one or more hardware processors configured by machine-readable instructions including a component that is the same as or similar to output component 116, in accordance with one or more implementations.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

1. A system configured to train and use machine learning models to predict subject responses to administered interventions based on results of clinical studies, the system comprising:
one or more physical processors configured by machine-readable instructions to:
obtain clinical information corresponding to a first study conducted on a first set of subjects in a real-world setting, wherein the first study involves administration of a first intervention to individual subjects of the first set of subjects, wherein the first study is conducted to evaluate the first intervention, wherein the clinical information includes responses by the individual subjects of the first set of subjects to being administered the first intervention, and wherein the clinical information is obtained electronically;
identify, by the one or more physical processors, subsets of individual subjects within the first set of subjects based on the responses, wherein individual subjects of a given subset share the same or similar response to being administered the first intervention, such that a first subset and a second subset are identified, the first subset including individual subjects having responses that are the same or similar to each other to being administered the first intervention and the second subset including different individual subjects having responses that are the same or similar to each other to being administered the first intervention, the responses of the subjects of the first subset being different and distinct from the responses of the subjects of the second subset;
obtain, by the one or more physical processors, subject information for the individual subjects of the first set of subjects that characterizes aspects of the individual subjects relevant to the responses to being administered the first intervention, such that the subject information for a given subject of the first set of subjects includes parameter values for one or more biological parameters related to a biological profile of the given subject and one or more historical parameters related to a medical history of the given subject, and wherein the subject information is obtained electronically;
based on the subject information and the identified subsets of individual subjects, identify, by the one or more physical processors, commonality of parameter values for individual parameters included in the subject information within the identified subsets, such that commonality of parameter values for a first parameter is identified for the first subset and commonality of parameter values for a second parameter is identified for the second subset;
train, by the one or more physical processors, a machine learning model to generate output that represents response predictions for subjects based on subject information for the subjects, wherein training the machine learning model includes training the machine learning model on input/output pairs that are based on the real-world setting of the first study, wherein the input/output pairs include a first input/output pair and a second input/output pair, the first input/output pair including the parameter values for the first parameter as input and the responses of the individual subjects of the first subset as output, and the second input/output pair including the parameter values for the second parameter as input and the responses of the individual subjects of the second subset as output;
obtain, by the one or more physical processors, secondary subject information for subjects of a second set of subjects, wherein the subjects of the second set of subjects have not been administered the first intervention, wherein the secondary subject information includes secondary parameter values, and wherein the secondary subject information is obtained electronically; and
generate, by the one or more physical processors, response predictions for the individual subjects of the second set of subjects by providing, to the trained machine learning model, the secondary subject information and the secondary parameter values as input, wherein the response predictions describe expected responses of the second set of subjects to being administered the first intervention, and wherein the trained machine learning model is configured to generate the response predictions as output such that:
(i) responsive to an individual subject of the second set of subjects having the secondary subject information that includes the secondary parameter values that are similar to the parameter values for the first parameter, the trained machine learning model generates an individual response prediction for the individual subject as output that is similar to the responses of the individual subjects of the first subset, and
(ii) responsive to the individual subject of the second set of subjects having the secondary subject information that includes the secondary parameter values that are similar to the parameter values for the second parameter, the trained machine learning model generates a different individual response prediction for the individual subject as output that is similar to the responses of the individual subjects of the second subset.

2. The system of claim 1, wherein the first intervention is a drug therapy, a medical device, a procedure, or a vaccine administered to individual subjects of the first set of subjects.

3. The system of claim 1, wherein biological parameters includes the age, sex, ancestry, phenotypes, and genotypes of the given subject.

4. The system of claim 1, wherein commonality of parameter values are defined by a range of values, such that the subject information for the individual subjects of the first subset includes values for the first parameter that are within a first range, and the subject information for the individual subjects of the second subset includes values for the second parameter that are within a second range.

5. The system of claim 1, wherein the subject information for a first subject including the commonality of parameter values for the first parameter is predictive of the first subject having a response to being administered the first intervention that is the same or similar to the individual subjects of the first subset, and wherein subject information for a second subject including the commonality of parameter values for the second parameter is predictive of the second subject having a response to being administered the first intervention that is the same or similar to the individual subjects of the second subset.

6. The system of claim 1, wherein the one or more processors are further configured by machine-readable instructions to:
identify subsets of the second set of subjects corresponding to the secondary subject information that includes the commonality of parameter values for parameters identified for the first set of subjects.

7. The system of claim 1, wherein training the machine learning model uses at least one of supervised learning, unsupervised learning and reinforcement learning.

8. The system of claim 1, wherein the one or more processors are further configured by machine-readable instructions to:
generate a clinical study report based on parameters for which commonality of parameter values within the subsets are identified, wherein the clinical study report describes the commonality of parameter values, the identified subsets, and the responses of individual subjects within the same subsets.

9. The system of claim 1, wherein the one or more physical processors are further configured by machine-readable instructions to:
aggregate the commonality of parameter values and the responses of subjects of the corresponding subsets into input/output pairs for training of a particular machine learning model.

10. The system of claim 1, wherein the one or more physical processors are further configured by machine-readable instructions to:
store the trained machine learning model.

11. A method for training and using machine learning models to predict subject responses to administered interventions based on results of clinical studies, the method comprising:
obtaining, by one or more physical processors, clinical information corresponding to a first study conducted on a first set of subjects in a real-world setting, wherein the first study involves administration of a first intervention to individual subjects of the first set of subjects, wherein the first study is conducted to evaluate the first intervention, wherein the clinical information includes responses by the individual subjects of the first set of subjects to being administered the first intervention, and wherein the clinical information is obtained electronically;
identifying, by the one or more physical processors, subsets of individual subjects within the first set of subjects based on the responses, wherein individual subjects of a given subset share the same or similar response to being administered the first intervention, including identifying a first subset and a second subset, the first subset including individual subjects having responses that are the same or similar to each other to being administered the first intervention and the second subset including different individual subjects having responses that are the same or similar to each other to being administered the first intervention, the responses of the subjects of the first subset being different and distinct from the responses of the subjects of the second subset;
obtaining, by the one or more physical processors, subject information for the individual subjects of the first set of subjects that characterizes aspects of the individual subjects relevant to the responses to being administered the first intervention, such that the subject information for a given subject of the first set of subjects includes parameter values for one or more biological parameters related to a biological profile of the given subject and one or more historical parameters related to a medical history of the given subject, and wherein the subject information is obtained electronically;
based on the subject information and the identified subsets of individual subjects, identifying, by the one or more physical processors, commonality of parameter values for individual parameters included in the subject information within the identified subsets, including identifying commonality of parameter values for a first parameter for the first subset and commonality of parameter values for a second parameter for the second subset;
training, by the one or more physical processors, a machine learning model to generate output that represents response predictions for subjects based on subject information for the subjects, wherein training the machine learning model includes training the machine learning model on input/output pairs that are based on the real-world setting of the first study, wherein the input/output pairs include a first input/output pair and a second input/output pair, the first input/output pair including the parameter values for the first parameter as input and the responses of the individual subjects of the first subset as output, and the second input/output pair including the parameter values for the second parameter as input and the responses of the individual subjects of the second subset as output;
obtaining, by the one or more physical processors, secondary subject information for subjects of a second set of subjects, wherein the subjects of the second set of subjects have not been administered the first intervention, wherein the secondary subject information includes secondary parameter values, and wherein the secondary subject information is obtained electronically; and
generating, by the one or more physical processors, response predictions for the individual subjects of the second set of subjects by providing, to the trained machine learning model, the secondary subject information and the secondary parameter values as input, wherein the response predictions describe expected responses of the second set of subjects to being administered the first intervention, and wherein the trained machine learning model generates the response predictions as output such that:
(i) responsive to an individual subject of the second set of subjects having the secondary subject information that includes the secondary parameter values that are similar to the parameter values for the first parameter, the trained machine learning model generates an individual response prediction for the individual subject as output that is similar to the responses of the individual subjects of the first subset, and
(ii) responsive to the individual subject of the second set of subjects having the secondary subject information that includes the secondary parameter values that are similar to the parameter values for the second parameter, the trained machine learning model generates a different individual response prediction for the individual subject as output that is similar to the responses of the individual subjects of the second subset.

12. The method of claim 11, wherein the first intervention is a drug therapy, a medical device, a procedure, or a vaccine administered to individual subjects of the first set of subjects.

13. The method of claim 11, wherein biological parameters includes the age, sex, ancestry, phenotypes, and genotypes of the given subject.

14. The method of claim 11, wherein commonality of parameter values are defined by a range of values, such that the subject information for the individual subjects of the first subset includes values for the first parameter that are within a first range, and the subject information for the individual subjects of the second subset includes values for the second parameter that are within a second range.

15. The method of claim 11, wherein the subject information for a first subject including the commonality of parameter values for the first parameter is predictive of the first subject having a response to being administered the first intervention that is the same or similar to the individual subjects of the first subset, and wherein subject information for a second subject including the commonality of parameter values for the second parameter is predictive of the second subject having a response to being administered the first intervention that is the same or similar to the individual subjects of the second subset.

16. The method of claim 11, wherein the method further comprises:
identifying subsets of the second set of subjects corresponding to the secondary subject information that includes the commonality of parameter values for parameters identified for the first set of subjects;

17. The method of claim 11, wherein access permissions are required to access the subject information for the first set of subjects and the secondary subject information of the second set of subjects.

18. The method of claim 11, wherein the method further comprises:
generating a clinical study report based on parameters for which commonality of parameter values within the subsets are identified, wherein the clinical study report describes the commonality of parameter values, the identified subsets, and the responses of individual subjects within the same subsets.

19. The method of claim 11, wherein the method further comprises:
aggregating the commonality of parameter values and the responses of subjects of the corresponding subsets into input/output pairs for training of a particular machine learning model.

20. The method of claim 11, wherein the method further comprises:
storing the trained machine learning model.
